(19) United States
(12) Patent Application Publication
(10) Pub. No.: US 2021/0030678 A1
(43) Pub. Date: Feb. 4, 2021

(54) CANNABINOID AND CBD LIPOSOME FORMULATIONS AND USES THEREOF

(71) Applicant: John Hartenbach, Irvine, CA (US)

(72) Inventor: John Hartenbach, Irvine, CA (US)

(73) Assignee: Medterra Pharma, Irvine, CA (US)

(21) Appl. No.: 16/984,058

(22) Filed: Aug. 3, 2020

Related U.S. Application Data
(60) Provisional application No. 62/881,756, filed on Aug. 1, 2019.

(51) Int. Cl. A61K 9/127; A61K 36/185; A61K 36/906; A61K 31/12; A61K 31/4045; A61K 33/06; A23L 33/105; A23L 33/16

(52) U.S. Cl. CPC A61K 9/1271; A61K 36/185; A61K 36/906; A61K 31/12; A61K 9/006; A61K 33/06; A23L 33/105; A23L 33/16; A61K 31/4045

(57) ABSTRACT

Aspects of the present application provide liposomal formulations of cannabinoid extracts. Method of using such formulation, e.g., for the treatment of inflammation are also provided.

### CROSS REFERENCE TO RELATED APPLICATION(S)

This application claims the benefit of priority of U.S. Provisional Application Ser. No. 62/881,756, titled “Cannabinoid And CBD Liposome Formulations And Uses Thereof” filed Aug. 1, 2019, the entire content is incorporated herein by reference.

### BACKGROUND OF THE INVENTION

### 1. Field of the Invention

The present invention relates generally to the field of medicine and the use pharmaceuticals and nutraceuticals. More particularly, it concerns cannabidiol formulations and method of their use.

### 2. Description of Related Art

Cannabidiol (CBD) has been recognized as a potential therapeutic agent for a variety of health conditions. However, until recently government regulation was a significant impediment to use of CBD products. Moreover, to date, formulations and method for efficient delivery of CBD have been lacking.

### SUMMARY OF THE INVENTION

Certain aspects of the present disclosure relate to a composition comprising a liposomal formulation of cannabinoid extract, wherein said composition is essentially free from tetrahydrocannabinol (THC) components. In particular aspects, the cannabinoid extract further comprises cannabidivarin (CBDV); Cannabinol (CBN); and/or cannabichromene (CBC). In specific aspects, the cannabinoid extract is composed of at least about 95% cannabidiol by weight. In more specific aspects, the cannabinoid extract is composed of at least about 96%, 97%, 98% or 99% cannabidiol by weight. In particular aspects, the cannabinoid extract comprises less than 1% or 0.5% CBDV by weight. In some aspects, the cannabinoid extract comprises less than 0.1% or 0.05% CBC by weight.

In certain aspects, the cannabinoid extract is from a hemp extract. In particular aspects, the hemp extract is from non-GMO hemp. In specific aspects, the composition is sterile. In particular aspects, the composition is formulated in a nutraceutical formulation or in a pharmaceutical formulation. In some aspects, the composition is formulated for oral, sublingual, ocular or topical administration. In specific aspects, the liposomal formulation comprises a phospholipid, and in even more specific aspects the phospholipid is phosphocholine (PC), egg PC, soybean PC, DPPC or DOPC.

In particular aspects, the liposomes of the liposomal formulation are composed of at least 70%, 75%, 80% or 85% PC. In certain aspects, the liposomes comprise a cholesterol component. In some aspects, the liposomes of the liposomal formulation further comprise a protein component, BSA, lithocholic acid and/or an lecithin (e.g., sunflower lecithin). In certain aspects, the liposomal formulation is dissolved in a substantially aqueous carrier. In specific aspects, the aqueous carrier comprises water, fruit juice, vegetable juice or an alcohol. In particular aspects, the liposomes of the liposomal formulation have an average diameter of between 50 nm and 500 nm, or more particularly between 50 nm and 200 nm, or more particularly between 80 nm and 100 nm, or even more particularly between 85 nm and 95 nm. In certain aspects, a liposome of the embodiments is 1 μm or larger such as about 1 to 10 μm, 5 to 50 μm or 10 to 100 μm. In certain aspects, the liposomes of the liposomal formulation have an average diameter of between 80 nm and 100 nm and are composed of at least 60%, 75%, 80%, 85% or 90% PC.

In particular aspects, the composition further comprises curcumin and/or ginger extracts, and in specific aspects the curcumin and/or ginger extracts are comprised in the liposomes. In some aspects, the composition further comprises curcumin and ginger extracts. In specific aspects, the composition further comprises melatonin and/or magnesium. In particular aspects, the melatonin and/or magnesium are comprised in the liposomes. In certain aspects, the composition further comprises melatonin and/or magnesium. In specific aspects, the liposomal formulation comprises omega-3 fatty acids.

In the composition comprises an oil component, and in particular aspects the oil component comprises canola oil, flaxseed oil, rapeseed oil, soybean oil, walnut oil, fish oil, safflower oil, chia seed oil, sunflower seed oil, sesame seed oil, seaweed oil, corn oil, cotton seed oil, peanut oil, palm oil, avocado oil, coconut oil, or olive oil. In particular aspects, the composition is further defined as a herbal, vitamin or energy-providing nutraceutical beverage. Certain aspects further comprise a preservative, flavoring agent, dye, vitamin, anti-oxidant, or plant extract. In specific aspects, the composition is formulated as a cream for topical administration. Particular aspects include a sealed vial comprising a composition as disclosed herein, including for example, the composition of the embodiments detailed above. In specific aspects, the container has 0.5 ml to 10 ml of the composition.

Certain aspects include a method of improving the health or well-being of a subject comprising administering a composition as disclosed herein, including for example, a composition of the embodiments. Particular aspects include a method of treating a subject in need thereof comprising administering an effective amount of a composition according to the present disclosure, including for example, a composition in accordance of the embodiments detailed above. In specific aspects, the method is further defined as a method treating inflammation in a subject. In particular aspects, the inflammation is skin inflammation, wound inflammation, cardiovascular inflammation, neurological inflammation, liver inflammation or gut inflammation.

In certain aspects, the method is further defined as a method for treating Crohn's disease, inflammatory bowel disease. In particular aspects, the method is further defined as a method for treating or preventing dementia or Alzheimer's disease. In specific aspects, the method is further defined as a method for treating or preventing atherosclerosis, stroke, heart failure or cardiac hypertrophy. In certain aspects, the administration is effective to reduce levels of pro-inflammatory cytokines in the subject. In particular aspects, the administration is by topical administration of a cream and is effective to reduce levels of pro-inflammatory cytokines in the blood of the subject. In specific aspects, the administration is effective to increase IL-10 levels, reduce neutrophil accumulation, reduce IL-10 levels and/or reduce TNFα levels in the subject.

In certain aspects, the subject is a human. In particular aspects, the composition is administered weekly, daily, twice a day, three times a day, every six hours, every three hours or hourly. In specific aspects, the composition is administered over the period of a week, two weeks, a month or a year. In particular aspects, the method is further defined as a method for treating or preventing a neurological disease or neurological injury in the subject. In certain aspects, the neurological disease is Alzheimer's disease. In specific aspects, the method is further defined as a method of reducing beta-amyloid levels in the subject. In particular aspects, the method is further defined as a method for treating or preventing the progression of Alzheimer's disease is a subject. In certain aspects, the subject is at risk for developing Alzheimer's disease, and in specific aspects, the subject has or is diagnosed with a genetic predisposition for Alzheimer's disease. In particular aspects, the neurological injury is a stroke. In certain aspects, the composition is administered topically. In specific aspects, the composition is administered orally or sublingually.

As used herein, “essentially free,” in terms of a specified component, is used herein to mean that none of the specified component has been purposefully formulated into a composition and/or is present only as a contaminant or in trace amounts. The total amount of the specified component resulting from any unintended contamination of a composition is preferably below 0.01%. Most preferred is a composition in which no amount of the specified component can be detected with standard analytical methods.

As used herein in the specification and claims, “a” or “an” may mean one or more. As used herein in the specification and claims, when used in conjunction with the word “comprising”, the words “a” or “an” may mean one or more than one. As used herein, in the specification and claim, “another” or “a further” may mean at least a second or more.

As used herein in the specification and claims, the term “about” is used to indicate that a value includes the inherent variation of error for the device, the method being employed to determine the value, or the variation that exists among the study subjects.

Other objects, features and advantages of the present invention will become apparent from the following detailed description. It should be understood, however, that the detailed description and the specific examples, while indicating certain embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1: Shows a graph illustrating the fold-difference of TNFα secretion in skin cells and immune cells involving no inflammatory trigger, an inflammatory trigger and an inflammatory trigger with CBD cream.

FIG. 2: Graph illustrates a global pathology score of treated skin for various conditions observed on mouse ears, including: (1) an inflammatory trigger without CBD cream; (2) an inflammatory trigger with CBD cream applied; (3) CBD cream without an inflammatory trigger; and (4) untreated.

FIG. 3: Graph shows neutrophil accumulation (myeloperoxidase) within inflamed tissue four hours after a triggering event for conditions including no stimulus, a topical inflammatory stimulus, and a topical inflammatory stimulus with topical CBD cream. Also shown is the relative TNFα in circulation for the same conditions.

FIG. 4: Graph shows circulating TNFα for a healthy mouse, inflamed condition, a CBD cream according to the present disclosure, and three different pain creams.

FIG. 5: Graph shows IL-10 production (Anti-Inflammatory Signal) for a healthy mouse, inflamed condition, a CBD cream according to the present disclosure, and three different pain creams.

FIG. 6: Graph shows inflammatory cytokines (TNFα and IL-6) associated with arthritis for a control, inflammatory stimulus, and CBD cream application. In this test, Human cells were treated ex vivo with an inflammatory agent (30 min) followed by CBD treatment (2 hrs). Cytokine secretion was then analyzed by flow cytometry-ELISA.

FIG. 7: Graph shows inflammatory cytokine TNFα for a healthy mouse, an inflamed condition, a CBD base according to the present disclosure, a CBD advanced formula according to the present disclosure, and three competitor pain creams.

FIG. 8: Graph shows inflammatory cytokine IL-1β for the conditions shown in FIG. 7 (i.e. a healthy mouse, an inflamed condition, a CBD base according to the present disclosure, a CBD advanced formula according to the present disclosure, and three competitor pain creams).

FIG. 9: Graph shows cytokine IL-10, for the conditions shown in FIG. 7 (i.e. a healthy mouse, an inflamed condition, a CBD base according to the present disclosure, a CBD advanced formula according to the present disclosure, and three competitor pain creams).

FIG. 10: A schematic illustration of the basic steps of the procedure used in the experimental setup.

FIG. 11: Graph showing the Geometric Mean of TNFα and percentage TNFα+ relative to inflammatory agent alone for conditions including an inflammatory agent, no inflammation, an inflammatory agent+CBD cream according to the present disclosure, and an inflammatory agent+advanced CBD cream according to the present disclosure.

FIG. 12: Graph shows responses for a healthy mouse, an inflammatory stimulus with CBD cream, and an inflammatory stimulus only, for increasing TNFα Concentration.

FIG. 13: Shows the substrate packaged in a proprietary liposomal delivery system according to the present disclosure that is more easily accessed and activated even in an in vitro system in which theoretically even naked substrate is 100% bioavailable. The lowest dose of packaged substrate tested was more active than the highest dose of naked substrate.

FIG. 14: Shows product diffusion strength for the naked product versus the packaged product.

FIG. 15: Graph shows the percentage of product-target reach relative to control versus time for the naked product and a liposomal product. Instead of being injected straight into the target, the products are now given orally to the mice, distal to the target site.

FIG. 16: Provides an image of naked product versus packaged product applied topically at 15 minute intervals reveals the naked product is relatively unable to penetrate through the skin at an effective rate, being washed off and more or less wasted. The product packaged in a delivery vehicle according to the present disclosure is now able to penetrate safely through the skin to the target where it will be much more effective.

FIG. 17: Graph shows the relative percentage of circulating TNFα versus time post inflammatory trigger for a control, CBD and packaged CBD application. As shown in the graph, while CBD is able to quell a systemic inflammatory event (measured in this experiment by relative concentrations of circulating TNFα), the CBD packaged in the liposomes is able to work quicker, longer, and more powerfully at equal doses.

FIG. 18: Tables show laboratory analyses for cannabinoid profile and potency; heavy metal analysis and microbiological contaminants of a cannabinoid extract of the embodiments.

FIG. 19: Graph shows liposome size distribution of a cannabinoid extract-liposome formulation of the embodiments.

### DESCRIPTION OF ILLUSTRATIVE EMBODIMENTS

### I. The Present Embodiments

Aspects of the present disclosure provide stable, fast-acting formulation of a cannabinoid or a cannabinoid analog. The formulation are encapsulated in liposomes to enhance bioavailability. In certain aspects, the such formulations can be used to treat inflammation. Studies here confirm that liposomal CBD formulations of the embodiments are effective in reducing even systemic markers of inflammation (such as pro-inflammatory cytokines).

Embodiments of the present disclosure generally include compositions comprising a liposomal formulation of a greater than 95% CBD cannabinoid extract extracted from industrial hemp, that is essentially free from tetrahydrocannabinol (THC) components. Additional embodiments may include cannabinoids such as cannabigerol (CBG), cannabidivarin (CBDV), Cannabinol (CBN) and/or cannabichromene (CBC) in addition to the CBD. The embodied compositions may be formulated in a nutraceutical or pharmaceutical formulation for oral, sublingual, ocular or topical administration.

The embodied liposomal formulations of the present disclosure generally include a phospholipid selected from phosphocholine (PC), egg PC, soybean PC, DPPC or DOPC; wherein the phosphocoline content of the liposomes of the liposomal formulation is at least 70%, 75%, 80% or 85% PC. Additionally, the liposomes may comprise a cholesterol component, a protein component, BSA and/or lithocholic acid.

In embodiments wherein the liposomal formulation is dissolved in a substantially aqueous carrier, the aqueous carrier may include water, fruit juice, vegetable juice or an alcohol.

Additional embodiments include the enhanced effects of various other ingredients including one or more individually or combined including but not limited to curcumin, ginger extracts, melatonin, magnesium, and/or omega-3 fatty acids.

In instances when the embodied composition comprises an oil component, the oil component may comprise any one or combination of canola oil, flaxseed oil, rapeseed oil, soybean oil, walnut oil, fish oil, safflower oil, chia seed oil, sunflower seed oil, sesame seed oil, seaweed oil, corn oil, cotton seed oil, peanut oil, palm oil, avocado oil, coconut oil, or olive oil.

The compositions of the present disclosure may further be used for improving the health or well-being of a subject by administering an effective amount in an effective route of administration.

### II. Formulations of the Embodiments

As used herein, the term “analog” refers to a compound that is structurally related to naturally occurring cannabinoids, but whose chemical and biological properties may differ from naturally occurring cannabinoids. In the present context, analog or analogs refer compounds that may not exhibit one or more undesirable side effects of a naturally occurring cannabinoid. In addition, analog refers to a compound that is derived from a naturally occurring cannabinoid by chemical, biological or a semi-synthetic transformation of the naturally occurring cannabinoid. Aspects of the present disclosure include liquid compositions of cannabinoids and their analogs. In addition, certain aspects also provide stable colloidal formulations that are manufactured by contacting a solution containing a cannabinoid, its analog, or both into a solvent such as water, with or without pharmaceutically acceptable buffers. A suitable solvent such as C1-C6 aliphatic alcohols or mixtures of water and alcohols, acetone or any water miscible organic solvent can be used to dissolve the cannabinoids.

In certain aspects, the inventive cannabinoid formulations are in the form of micelles or liposomes that encapsulate a cannabinoid or its analog within the membrane of the micelles or liposomes. Within the context of the present technology, the term “micelle” refers to an aggregate of surfactant molecules dispersed in a liquid colloid, while “liposome” refers to a vesicle composed of a mono or bilayer lipid.

Other drugs, and pharmaceutically acceptable carriers if present, may be in the lipophilic membrane or entrapped in the aqueous fluid that forms the core of the liposome. The entrapped cannabinoids contribute to the stability of the micelle/liposome membranes, such that the micelle/liposomes formulations may be used as an improved, fast, reliable and efficient system for the oral, enteral, parenteral, intravenous or topical delivery of cannabinoids and/or additional drugs to subjects in need thereof. As used herein, the term “subject” refers to a mammal subject to the delivery of the compositions disclosed herein. Mammalian subjects include without limitation humans, dog, cat, horse or any other animal subject to the delivery of the disclosed compositions.

Unilamellar micelles or liposomes that are thermostable at temperatures greater than 50° C. can be used in the manufacture of cannabinoid formulations according to the present disclosure. These micelles or liposomes can be obtained by contacting a solution of a cannabinoid, its analog or both (a cannabinoid extract), with an aqueous solvent or an aqueous solution of a pharmaceutically active compound or drug. The mixing of the cannabinoid solution occurs in a manner suitable for the rapid dissolution of the cannabinoid solution in the aqueous solution. This can be accomplished through a variety of means including dilution, injection through a small orifice under pressure, and/or ultrasonic atomization.

For certain embodiments, the inventive composition is in the form of a concentrated, stable colloidal suspension that is obtained by infusing a solvent solution containing the cannabinoid extract or pure cannabinoids into a solvent such as water, with or without buffer. A stabilizing agent, for instance, a polymer or compounds selected from cellulose hyaluronic acid, polyvinyl pyrrolidone (PVP), alginate, chondroitin sulfate, poly gamma glutamic acid, gelatin, chitisin, corn starch and flour can be used to stabilize the micelle formulations.

According to one aspect of the present disclosure, the maximum final concentration of a cannabinoids or an analog of the cannabinoid in the micellar colloidal suspension is from about 1.0 mg/mL to about 100.0 mg/mL both values inclusive. For some embodiments, the concentration of a cannabinoid extract within liposomes can be greater than about 1.0 mg/mL, about 2.0 mg/mL, about 3.0 mg/mL, 4.0 mg/mL, about 5.0 mg/mL, about 6.0 mg/mL, about 7.0 mg/mL, about 8.0 mg/mL, about 9.0 mg/mL, about 10.0 mg/mL, about 15.0 mg/mL, about 20.0 mg/mL, about 25 mg/mL or about 30.0 mg/mL. In some aspects, a liposomal composition of the embodiments comprises about 20.0 mg/mL to about 30.0 mg/mL of cannabinoid.

Typical concentrations of cannabinoids within a liposomal suspension according to the present disclosure are about 50 mg/mL. For certain embodiments, the maximum final concentration of cannabinoids or an analog of the cannabinoid in the liposomal formulation is from about 10.0 mg/mL to about 300.0 mg/mL both values inclusive, for example, about 15.0 mg/mL, about 20.0 mg/mL, about 30.0 mg/mL, about 40.0 mg/mL, about 50.0 mg/mL, about 60.0 mg/mL, about 70.0 mg/mL, about 80.0 mg/mL, about 90.0 mg/mL, about 150.0 mg/mL, about 200.0 mg/mL, about 250.0 mg/mL, or about 300.0 mg/mL.

In some cases, the size of liposomes can range from about 0.01 μm to about 2.0 μm. For certain embodiments, the size of the spherical micelles is about 0.05 μm, about 0.1 μm, about 0.15 μm, 0.2 μm, 0.25 μm, 0.3 μm, 0.35 μm, 0.4 μm, 0.45 μm, 0.5 μm, 0.55 μm, 0.6 μm, 0.7 μm, 0.75 μm, 0.8 μm, 0.85 μm, 0.9 μm, about 0.95 μm, about 1.0 μm, 1.20 μm, 1.40 μm, 1.50 μm, 1.60 μm, 1.70 μm, 1.80 μm, 1.90 μm and 2.0 μm. For certain embodiments, liposomes that are about 0.04 μm, about 0.05 μm, about 0.06 μm, about 0.07 μm, about 0.08 μm, or about 0.09 μm are used to formulate compositions of the embodiments.

The formulations of the present disclosure are therefore particularly suitable for oral administration and may be administered to subjects with a pre-existing condition or pre-disposed to certain disease conditions, acute pain, or chronic pain conditions. Conditions contemplated by the disclosure include, but are not limited to, gastrointestinal, metabolic, neurological, circulatory, soft tissue, musculoskeletal, chronic or acute pain, nausea, decreased appetite, skin disorders, sexual dysfunction, glaucoma, AIDS wasting, neuropathic pain, treatment of spasticity associated with multiple sclerosis, fibromyalgia, chemotherapy-induced nausea, allergies, inflammation, infection, epilepsy, depression, migraine, bipolar disorders, anxiety disorder, dependency and withdrawal. In addition, the methods of the disclosure may be used to alleviate or relief symptoms or side effects associated with anti-retroviral therapy, chemotherapy and radiation therapy.

As used herein, cannabinoid compounds include without limitation cannabinol, cannabidiol, Δ9-tetrahydrocannabinol, Δ8-tetrahydrocannabinol, 11-hydroxy-tetrahydrocannabinol, 11-hydroxy-Δ9-tetrahydrocannabinol, levonantradol, Δ11-tetrahydrocannabinol, tetrahydrocannabivarin, dronabinol, amandamide, nabilone, any combination thereof, any natural or synthetic modification thereof, or any natural or synthetic molecule with a basic cannabinoid structure. In one preferred embodiment, the cannabinoid is tetrahydrocannabinol (THC).

Natural cannabinoid compounds used in the inventive compositions may be readily obtained from plant tissue, for example, trichones of the C. sativa plant, by suspending the tissue in an appropriate solvent to extract cannabinoid compounds and other tissue components. Analytical purification of such an extract provides pharmaceutical grade cannabinoid compounds. Cannabinoid compounds may also be extracted from plant tissue under supercritical conditions. Solvents used for supercritical extraction of cannabinoids include without limitation carbon dioxide, or other gases in isolation or combination with or without solvent modifers, selected from ethanol, propanol, butanol, hexane, chloroform, dichloromethane, acetone, or any organic solvent capable of extracting cannabinoids, and alcohol-water mixtures, for instance water-ethanol or water-butanol mixtures.

In addition to natural cannabinoids, aspects of the present disclosure encompass synthetic cannabinoid compounds as well as cannabinoids and their analogs that are obtained using semi-synthetic protocols. The manufacture of cannabinoid compounds and their analogs using semi-synthetic means involves contacting an appropriate substrate with one of the cannabinoid synthase enzymes. In one example, tetrahydrocannabinolic acid (THCA) or its analogs can be manufactured semi-synthetically by contacting cannabigerolic acid (CBGA) or an appropriately substituted derivative of CBGA with THC synthase to obtain the corresponding THCA or THCA analog respectively. The compositions may also contain natural or synthetically modified cannabinoids.

The compositions according to the present disclosure have advantageous properties. For instance, micellar and liposomal compositions according to the present invention are stable at high temperatures (e.g. exceeding 50° C.), are stable to sonication, capable of carrying large payloads of cannabinoids as well as other drug suitable for use in combination therapy and can be stored for extended periods of time (e.g. greater than 20 weeks at 25° C.).

The compositions disclosed herein also exhibit superior systemic delivery and release of cannabinoids from the micelle or liposomes used in the manufacture of the disclosed composition. The release of a cannabinoid from a liposome or micelle of the disclosed compositions can be modulated by changing the ratio of the concentration of lipid to the concentration of cannabinoid present in the liposome.

In one embodiment, tissue specific delivery can be achieved by modifying the surface of the liposomes or micelles with compounds that bind specifically to biological macromolecules expressed on cellular surfaces. For instance, the micelle or liposomal surface can be derivatized to display an antibody specific to an antigen expressed on cancer cells.

According to one embodiment, compositions of the present disclosure are used in the treatment of disease conditions. For instance, a composition of the cannabinoid extract, (cannabinoid, an analog of a cannabinoid, or both), can be administered to a patient or subject in need of treatment either alone or in combination with other compounds/drugs having similar or different biological activities.

For example, compositions of the present disclosure may be administered in a combination therapy, i.e., either simultaneously in single or separate dosage forms or in separate dosage forms within hours or days of each other. Examples of compounds/drugs used in such combination therapies include without limitation, chemotherapeutic agents, immunosuppressive agents, immunostimulatory, anti-pyretic, cytokines, opioids, cytokines, cytotoxic agents, nucleolytic compounds, radioactive isotopes, receptors, pro-drug activating enzymes, which may be naturally occurring or produced by recombinant methods, anti-inflammatory agents, antibiotics, protease inhibitors, growth factors, osteo-inductive factors and the like.

In some embodiments, the composition further contains, in accordance with accepted practices of pharmaceutical compounding, one or more pharmaceutically acceptable excipients, diluents, adjuvants, stabilizers, emulsifiers, preservatives, colorants, buffers, flavor imparting agents. As previously stated, the compositions disclosed herein may contain a cannabinoid, an analog of a cannabinoid, or both and may be consumed directly or formulated into nutraceutical or pharmaceutically acceptable compositions suitable for oral, enteral, parenteral, intravenous or topical administration.

The term parenteral as used herein includes subcutaneous injections, intravenous, intramuscular, intrasternal injection or infusion techniques. Such excipients are well known in the art. Dosage forms for oral administration include food, beverages, drinks, soups, baked goods, syrups, oral pharmaceutical compositions, nutraceutical formulations, and the like. Suitable pharmaceutical carriers include any such materials known in the art, e.g., any liquid, gel, solvent, liquid diluent, solubilizer, polymer or the like, which is non-toxic and which does not significantly interact with other components of the formulations in a deleterious manner.

Liquid dosage forms for oral administration include, but are not limited to, pharmaceutically acceptable emulsions, solutions, suspensions, syrups and elixirs. In addition to the cannabinoid extract, the liquid dosage forms can contain inert diluents commonly used in the art. For example, liquid formulations can contain water, alcohol, polyethylene glycol ethers, or any other pharmaceutically acceptable solvents. Solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethyl formamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof may also be present in the inventive compositions. Additionally, oral compositions can include adjuvants such as wetting agents, emulsifying and suspending agents, sweetening, flavoring, and perfuming agents. When formulated as a suspension, the compositions disclosed herein contain the cannabinoid extract and suspending agents, for example, ethoxylated isostearyl alcohols, polyoxyethylene sorbitol, sorbitan esters, microcrystalline cellulose, aluminum metahydroxide, bentonite, agar-agar, tragacanth, and mixtures thereof.

Solid dosage forms suitable for oral administration include, capsules, tablets, pills, powders, and granules. In such solid dosage forms, the cannabinoid extract, for instance, a cannabinoid or a cannabinoid analog can be used alone or in combination with one or more drugs are mixed with at least one pharmaceutically acceptable excipient or carrier such as sodium citrate or dicalcium phosphate and/or: (a) fillers or extenders such as starches, lactose, sucrose, glucose, mannitol, and silicic acid; (b) binders such as, for example, carboxymethylcellulose, alginates, gelatin, polyvinylpyrrolidone, sucrose, and acacia; (c) humectants such as glycerol; (d) disintegrating agents such as agar-agar, calcium carbonate, potato or tapioca starch, alginic acid, certain silicates, and sodium carbonate; (e) solution retarding agents such as paraffin; (f) absorption accelerators such as quaternary ammonium compounds; (g) wetting agents such as, for example, acetyl alcohol and glycerol monostearate; (h) absorbents such as kaolin and bentonite clay; and (i) lubricants such as talc, calcium stearate, magnesium stearate, solid polyethylene glycols, sodium lauryl sulfate, and mixtures thereof. For capsules, tablets and pills, the dosage form can also comprise buffering agents.

Micellular or liposomal suspensions can be encapsulated with a variety of polymers, sugars, and chelating agents, to yield stable solid liposomal cannabinoid preparation. Encapsulation can take the form of cross-linked polymers, trapping of the micelles or liposomes within a non-crosslinked polymer network; or dispersed within the crystalline structure of sugar starches or protein molecules. These granular can be further process to yield sublingual films, suppositories, dispersible powder, tablets, gel capsules, etc.

Solid dosages in the form of tablets, dragees, capsules, pills, and granules can be coated using compounds that accelerate or decrease the release of cannabinoids. For example, embodiments of the present disclosure can encompass solid dosage forms having enteric coatings, extended-release coatings, sustained-release coatings, delayed release coatings and immediate-release coatings. Methods used to coat solid dosage forms as well as the materials used to manufacture such coatings are well known in the pharmaceutical formulary art. The solid dosage forms can optionally contain opacity enhancing agents. According to an embodiment, the solid dosage form comprises an enteric coating that permits the release of a cannabinoid or cannabinoid analog alone or in combination with one or more drugs at a specific location within the gastrointestinal tract, optionally, in a delayed manner. Exemplary of such coating materials include glyceryl monostearate or glyceryl distearate may be employed, polymeric substances and waxes. The cannabinoid extract, for instance, a cannabinoid or cannabinoid analog alone or in combination with one or more drugs can also be in micro-encapsulated form, if appropriate, with one or more of the above-mentioned excipients.

A dietary composition according to the present invention is any ingestible preparation that contains the cannabinoid suspensions of the invention mixed with a food product. The food product can be dried, cooked, boiled, lyophilized or baked. Breads, teas, soups, cereals, salads, sandwiches, sprouts, vegetables, animal feed, pills and tablets, are among the vast number of different food products contemplated in the present invention.

Compositions for parenteral injection comprise pharmaceutically acceptable sterile aqueous or nonaqueous solutions, dispersions, suspensions or emulsions as well as sterile powders for reconstitution into sterile injectable solutions or dispersions prior to use. Examples of suitable aqueous and nonaqueous carriers, diluents, solvents or vehicles include water, ethanol, polyols (such as glycerol, propylene glycol, polyethylene glycol, and the like), carboxymethylcellulose and suitable mixtures thereof, vegetable oils (such as olive oil), and injectable organic esters such as ethyl oleate. Proper fluidity can be maintained, for example, by the use of coating materials such as lecithin (e.g., sunflower lecithin), by the maintenance of the required particle size in the case of dispersions, and by the use of surfactants. The compositions of the present disclosure can also contain adjuvants such as, but not limited to, preservatives, wetting agents, emulsifying agents, and dispersing agents. Compositions for parenteral delivery generally include isotonic agents such as sugars, sodium chloride, and the like. Prolonged absorption of the injectable pharmaceutical formulation can be brought about by the inclusion of agents which delay absorption such as aluminum monostearate and gelatin.

Injectable depot forms are made by forming microencapsule matrices of the drug in biodegradable polymers such as polylactide-polyglycolide. Depending upon the ratio of drug to polymer and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions which are compatible with body tissues. The injectable formulations can be sterilized, for example, by filtration through a bacterial-retaining filter, or by incorporating sterilizing agents in the form of sterile solid compositions which can be dissolved or dispersed in sterile water or other sterile injectable medium just prior to use.

Dosage forms for topical administration include, but are not limited to, ointments, creams, emulsions, lotions, gels, sunscreens and agents that favor penetration within the epidermis. Various additives, known to those skilled in the art, may be included in the topical formulations of the invention. Examples of additives include, but are not limited to, solubilizers, skin permeation enhancers, preservatives (e.g., anti-oxidants), moisturizers, gelling agents, buffering agents, surfactants, emulsifiers, emollients, thickening agents, stabilizers, humectants, dispersing agents and pharmaceutical carriers. Examples of moisturizers include jojoba oil and evening primrose oil. Suitable skin permeation enhancers are well known in the art and include lower alkanols, such as methanol ethanol and 2-propanol; alkyl methyl sulfoxides such as dimethylsulfoxide (DMSO), decylmethylsulfoxide (C10 MSO) and tetradecylmethyl sulfoxide; pyrrolidones, urea; N,N-diethyl-m-toluamide; C2-C6 alkanediols; dimethyl formamide (DMF), N,N-dimethylacetamide (DMA) and tetrahydrofurfuryl alcohol. Examples of solubilizers include, but are not limited to, hydrophilic ethers such as diethylene glycol monoethyl ether (ethoxydiglycol, available commercially as Transcutol®) and diethylene glycol monoethyl ether oleate (available commercially as Softcutol®); polyoxy 35 castor oil, polyoxy 40 hydrogenated castor oil, polyethylene glycol (PEG), particularly low molecular weight PEGs, such as PEG 300 and PEG 400, and polyethylene glycol derivatives such as PEG-8 caprylic/capric glycerides (available commercially as Labrasol®); alkyl methyl sulfoxides, such as DMSO; pyrrolidones, DMA, and mixtures thereof.

Prevention and/or treatment of infections can be achieved by the inclusion of antibiotics, as well as various antibacterial and antifungal agents, for example, paraben, chlorobutanol, phenol sorbic acid, and the like, in the compositions of the present disclosure.

One of ordinary skill will appreciate that effective amounts of the agents in the compositions used in the methods of the present disclosure can be determined empirically. It will be understood that, when administered to a human patient, the total daily usage of the composition of the present disclosure will be decided by the attending physician within the scope of sound medical judgment. The specific therapeutically effective dose level for any particular patient will depend upon a variety of factors: the type and degree of the response to be achieved; the activity of the specific composition employed; the age, body weight, general health, sex and diet of the patient; the duration of the treatment; drugs used in combination or coincidental with the method of the invention; and like factors well known in the medical arts.

The potential commercial uses of the disclosed preparations include, for example, protective/prophylactic and medical uses. The compositions of the present disclosure can also be administered by a variety of other routes, including mucosal, subcutaneous and intramuscular administration, and may comprise a variety of carriers or excipients known in the formulary art, such as, non-toxic solid, semisolid or liquid filler, diluent, encapsulating material and formulation auxiliaries that are pharmaceutically acceptable.

Embodiments of the present disclosure thus generally described, will be understood more readily by reference to the following examples, which are provided by way of illustration only, and are not intended to be limit the scope of the present disclosure.

### III. Examples

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

Examples of Non-Limiting Scientific Studies Performed to Exemplify the Activities of the Embodied Methods of Treating and Compositions Used for Treatment of Various Physiological Conditions of Events.

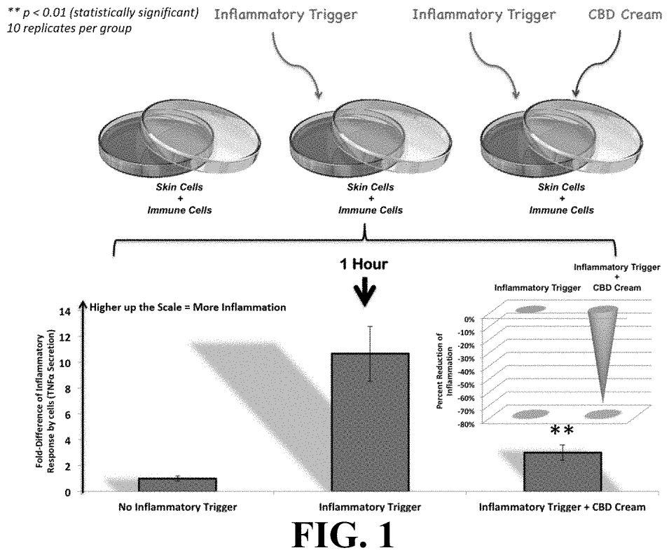

FIG. 1 includes a graph illustrating the fold-difference of inflammatory response by cells (TNFα secretion) for cases with skin cells and immune cells involving no inflammatory trigger, an inflammatory trigger and an inflammatory trigger with CBD cream.

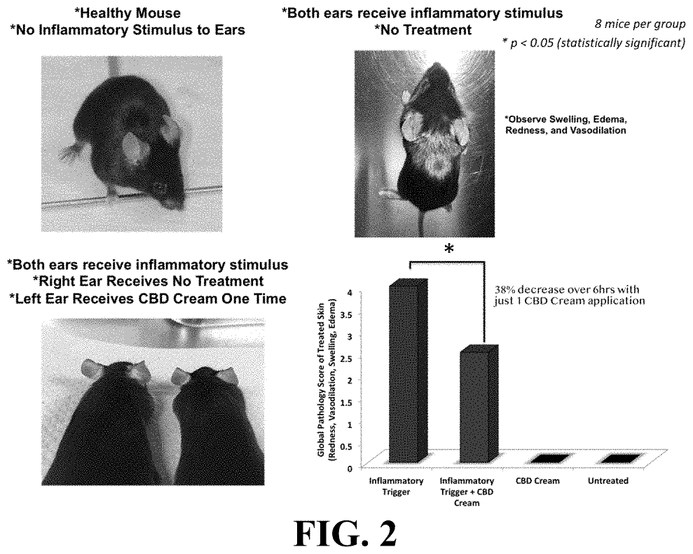

FIG. 2 includes a graph illustrating a global pathology score of treated skin for various conditions observed on mouse ears, including: (1) an inflammatory trigger without CBD cream; (2) an inflammatory trigger with CBD cream applied; (3) CBD cream without an inflammatory trigger; and (4) untreated.

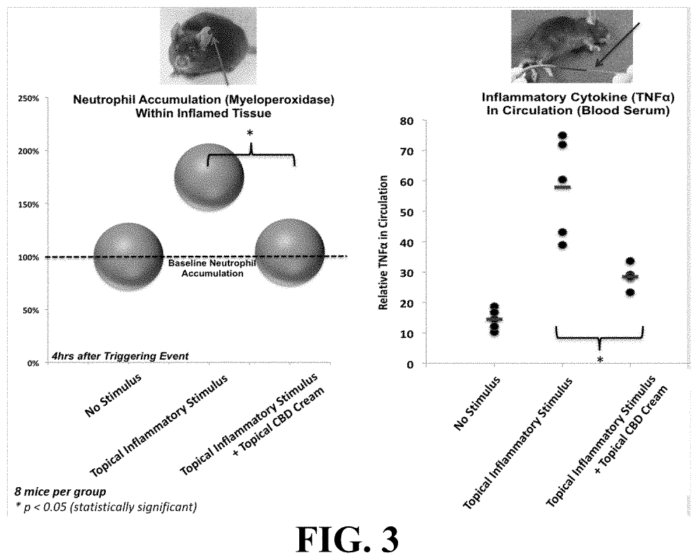

As shown in FIG. 3, a graph shows neutrophil accumulation (myeloperoxidase) within inflamed tissue four hours after a triggering event for conditions including no stimulus, a topical inflammatory stimulus, and a topical inflammatory stimulus with topical CBD cream. In addition, FIG. 3 shows the relative TNFα in circulation for the same conditions.

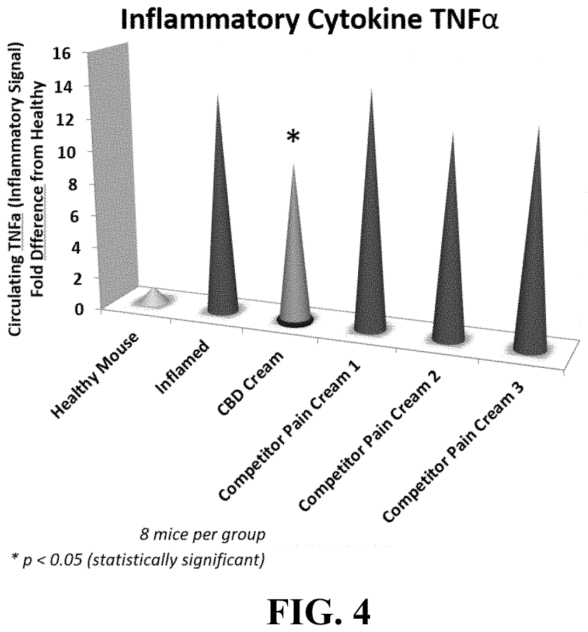

FIG. 4 includes a graph illustrating Circulating TNFα (Inflammatory Signal) for a healthy mouse, inflamed condition, a CBD cream according to the present disclosure, and three different pain creams. TNFα is a well-known and well-studied pro-inflammatory cytokine that is secreted during a variety of inflammatory events, and subsequently is the focus of many prescription anti-inflammatory medications [1-4]. Many “pain creams” only temporarily mask the pain, but do nothing to alter the underlying physiological issues.

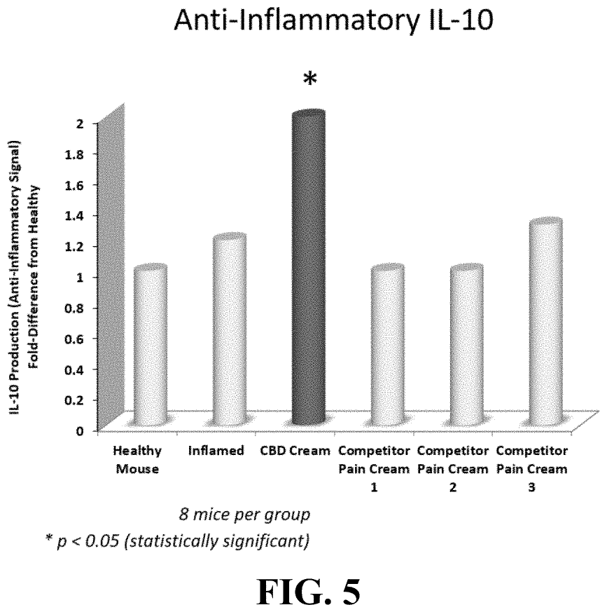

Referring now to FIG. 5, a graph is shown illustrating IL-10 Production (Anti-Inflammatory Signal) for a healthy mouse, inflamed condition, a CBD cream according to the present disclosure, and three different pain creams. In the data shown, competitor pain cream 1 is Ben Gay, competitor pain cream 2 is Capzasin-HP Arthritis Cream, and competitor pain cream 3 is Sportscreme Deep Penetrating Pain Relieving Rub. While it would be beneficial “turn down” inflammatory players like TNFα, it would be exponentially beneficial for a true pain cream to also turn on anti-inflammatory agents like IL-10 that are known to suppress immune responses and limit damaging effects. [5-8].

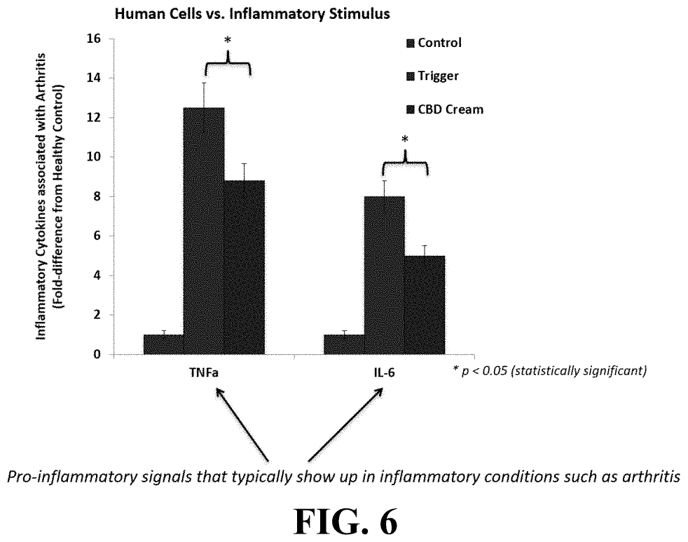

FIG. 6 includes a graph illustrating inflammatory cytokines (TNFα and IL-6) associated with arthritis for a control, inflammatory stimulus, and CBD cream application. In this test, Human cells were treated ex vivo with an inflammatory agent (30 min) followed by CBD treatment (2 hrs). Cytokine secretion was then analyzed by flow cytometry-ELISA.

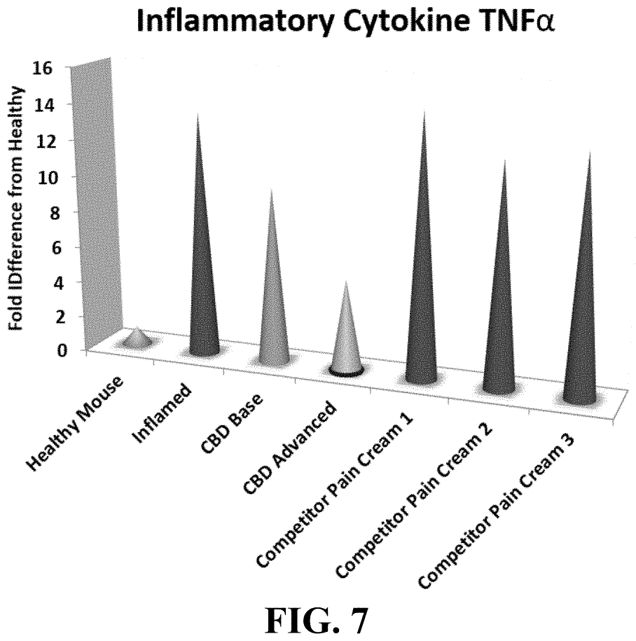

Referring now to FIG. 7, a graph illustrates inflammatory cytokine TNFα for a healthy mouse, an inflamed condition, a CBD base according to the present disclosure, a CBD advanced formula according to the present disclosure, and three competitor pain creams.

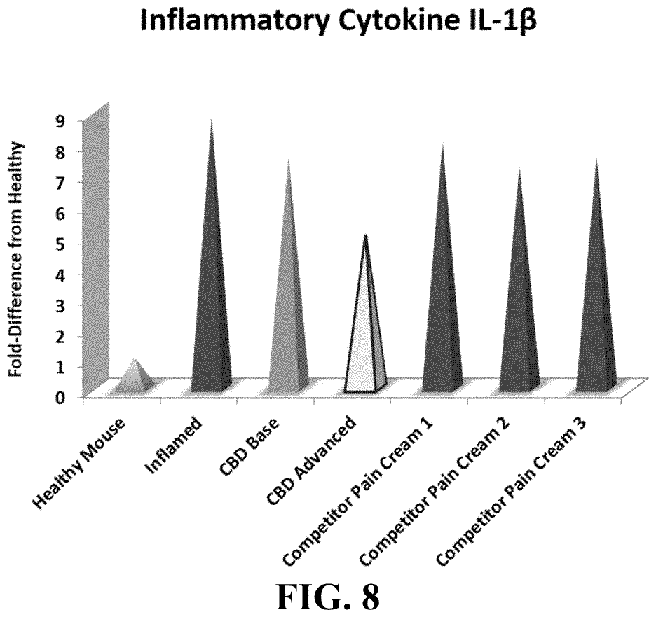

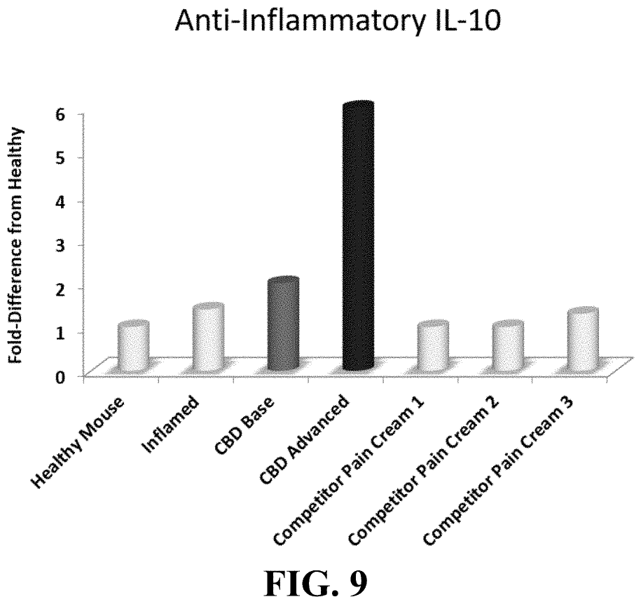

FIGS. 8 and 9 include a graph illustrating inflammatory cytokine IL-1β and IL-10, respectively, for the conditions shown in FIG. 7 (i.e. a healthy mouse, an inflamed condition, a CBD base according to the present disclosure, a CBD advanced formula according to the present disclosure, and three competitor pain creams). Il-1β is a classical pro-inflammatory cytokine that aids in prolonging tissue damage and upregulating other pro-inflammatory cytokines, and thus is also a focus of therapeutic intervention [9-12].

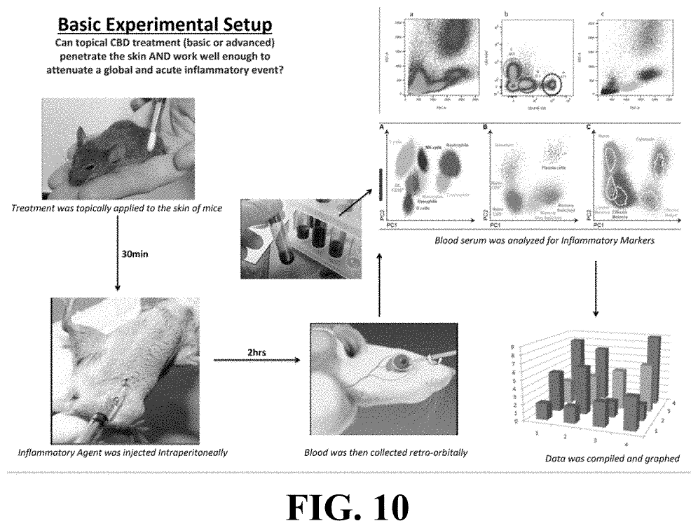

FIG. 10 illustrates the basic steps of the procedure used in the experimental setup. Treatment was first applied topically to the skin of the mice. After 30 minutes, an inflammatory agent was injected intraperitoneally. After two hours, blood was collected from the mice retro-orbitally. The blood serum was then analyzed for inflammatory markers. Finally, the data was compiled and graphed.

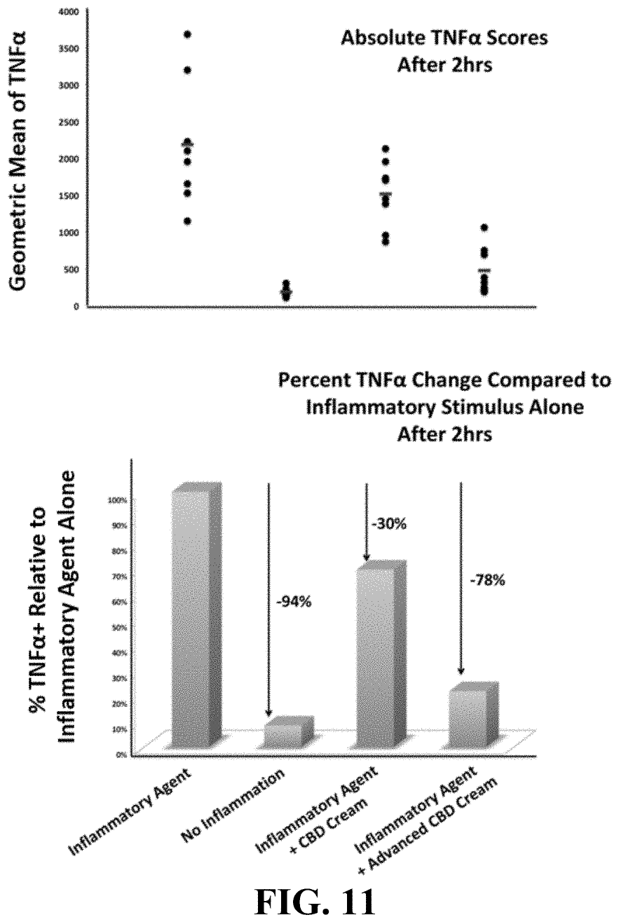

FIG. 11 includes a graph illustrating the Geometric Mean of TNFα and percentage TNFα+ relative to inflammatory agent alone for conditions including an inflammatory agent, no inflammation, an inflammatory agent+CBD cream according to the present disclosure, and an inflammatory agent+advanced CBD cream according to the present disclosure.

In conclusion, the experimental system worked (e.g. systemic inflammation was induced and able to be analyzed in circulating blood serum). CBD cream according to the present disclosure does possess anti-inflammatory properties and was able to penetrate the skin at rate X to reduce systemic inflammation. The advanced formula according to the present disclosure was able to penetrate the skin and/or reduce inflammation in a more pronounced way than the standard CBD cream according to the present disclosure. Due to either enhanced penetration and/or effectiveness, the advanced CBD cream could be the best anti-inflammatory CBD cream on the market, even able to significantly attenuate total body chronic inflammation.

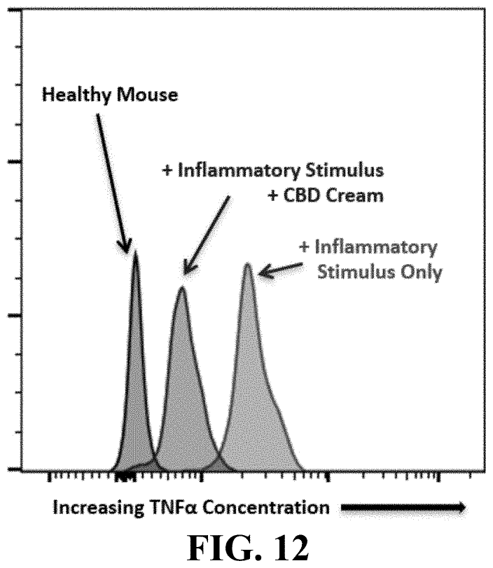

Referring now to FIG. 12, a graph illustrates responses for a healthy mouse, an inflammatory stimulus with CBD cream, and an inflammatory stimulus only, for increasing TNFα Concentration.

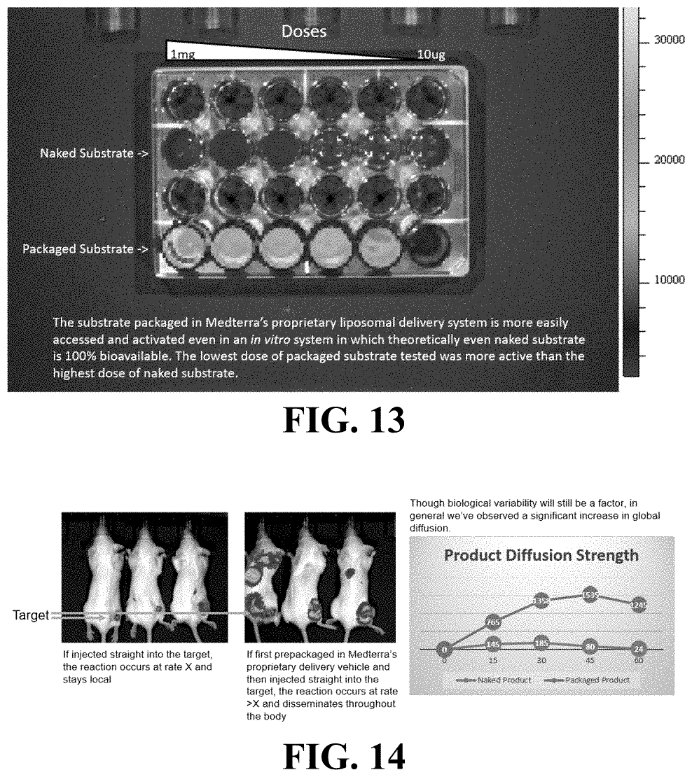

FIG. 13 illustrates the substrate packaged in a proprietary liposomal delivery system according to the present disclosure that is more easily accessed and activated even in an in vitro system in which theoretically even naked substrate is 100% bioavailable. The lowest dose of packaged substrate tested was more active than the highest dose of naked substrate.

FIG. 14 illustrates product diffusion strength for the naked product versus the packaged product. As shown in the figure, if injected straight into the target, the reaction occurs at rate X and stays local. However, if first prepackaged in the delivery vehicle according to the present disclosure and then injected straight into the target, the reaction occurs at rate >X and disseminates throughout the body. The increased potency and absorptive/diffusion rates means that the ‘desired effects’ can be achieved at much lower doses (possibly >10 times less), and lowering the dose of any ‘supplement’ taken is an advantage as the body (e.g. liver, kidneys) is taxed less and benefitted more.

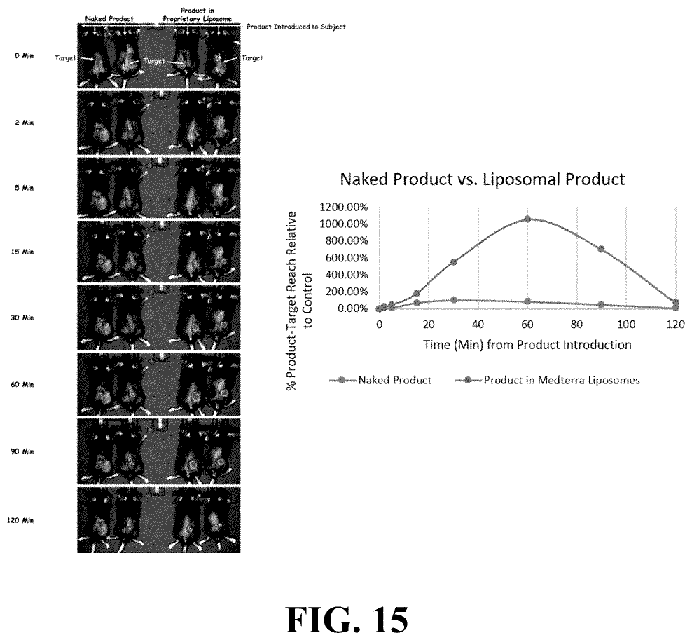

Referring now to FIG. 15, a graph illustrates the percentage of product-target reach relative to control versus time for the naked product and a liposomal product. Instead of being injected straight into the target, the products are now given orally to the mice, distal to the target site.

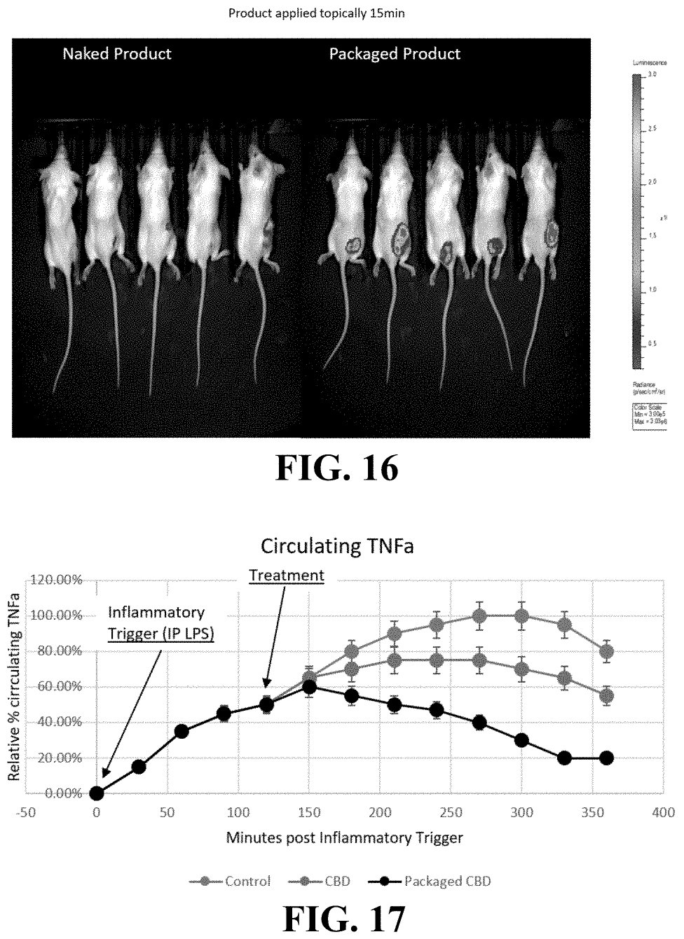

As shown in FIG. 16 an image of naked product versus packaged product applied topically at 15 minute intervals reveals the naked product is relatively unable to penetrate through the skin at an effective rate, being washed off and more or less wasted. The product packaged in a delivery vehicle according to the present disclosure is now able to penetrate safely through the skin to the target where it will be much more effective.

FIG. 17 includes a graph illustrating the relative percentage of circulating TNFα versus time post inflammatory trigger for a control, CBD and packaged CBD application. As shown in the graph, while CBD is able to quell a systemic inflammatory event (measured in this experiment by relative concentrations of circulating TNFα), the CBD packaged in the liposomes is able to work quicker, longer, and more powerfully at equal doses.

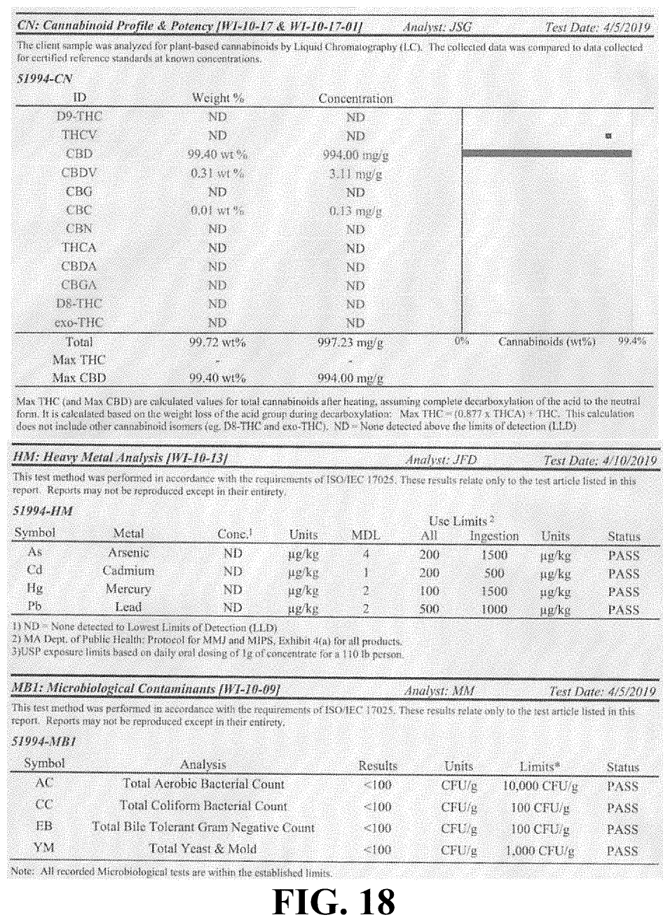

FIG. 18 shows lab analyses for cannabinoid profile and potency; heavy metal analysis and microbiological contaminants.

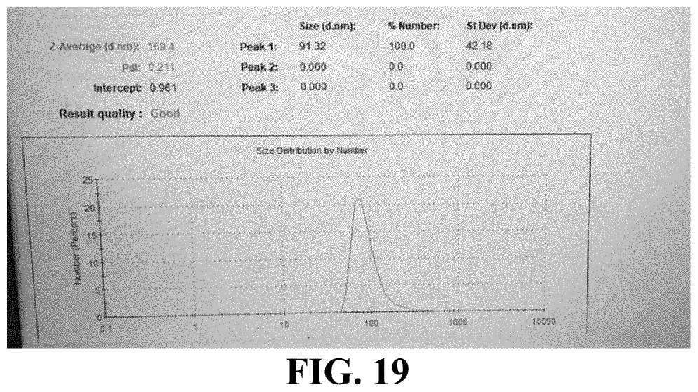

FIG. 19 includes a graph that shows liposome size distribution and the CBD extract composition.

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
1. Bradley, J. R. TNF-mediated inflammatory disease. J Pathol 214, 149-160 (2008).
2. Esposito, E. & Cuzzocrea, S. TNF-alpha as a therapeutic target in inflammatory diseases, ischemia-reperfusion injury and trauma. Curr Med Chem 16, 3152-3167 (2009).
3. Page, M. J., Bester, J. & Pretorius, E. The inflammatory effects of TNF-alpha and complement component 3 on coagulation. Sci Rep 8, 1812 (2018).
4. Popa, C., Netea, M. G., van Riel, P. L., van der Meer, J. W. & Stalenhoef, A. F. The role of TNF-alpha in chronic inflammatory conditions, intermediary metabolism, and cardiovascular risk. J Lipid Res 48, 751-762 (2007).
5. Cianciulli, A. et al. IL-10 plays a pivotal role in anti-inflammatory effects of resveratrol in activated microglia cells. Int Immunopharmacol 24, 369-376 (2015).
6. Couper, K. N., Blount, D. G. & Riley, E. M. IL-10: the master regulator of immunity to infection. J Immunol 180, 5771-5777 (2008).
7. Gutierrez-Murgas, Y. M., Skar, G., Ramirez, D., Beaver, M. & Snowden, J. N. IL-10 plays an important role in the control of inflammation but not in the bacterial burden in S. epidermidis CNS catheter infection. J Neuroinflammation 13, 271 (2016).
8. Ip, W. K. E., Hoshi, N., Shouval, D. S., Snapper, S. & Medzhitov, R. Anti-inflammatory effect of IL-10 mediated by metabolic reprogramming of macrophages. Science 356, 513-519 (2017).
9. Dinarello, C. A. Interleukin-1 in the pathogenesis and treatment of inflammatory diseases. Blood 117, 3720-3732 (2011).
10. Mayer-Barber, K. D. & Yan, B. Clash of the Cytokine Titans: counter-regulation of interleukin-1 and type I interferon-mediated inflammatory responses. Cell Mol Immunol 14, 22-35 (2017).
11. Ren, K. & Tones, R. Role of interleukin-1beta during pain and inflammation. Brain Res Rev 60, 57-64 (2009).
12. Rider, P. et al. IL-1alpha and IL-1beta recruit different myeloid cells and promote different stages of sterile inflammation. J Immunol 187, 4835-4843 (2011).

What is claimed is:

1. A composition comprising a liposomal formulation of a cannabinoid extract;
wherein said composition is formulated in a nutraceutical or pharmaceutical formulation; formulated for oral, sublingual, ocular or topical administration;
wherein said liposomal formulation is dissolved in a substantially aqueous carrier; and
wherein the liposomal formulation comprises a phospholipid, and further wherein the phospholipid comprises at least 70% of one or more phosphocholine sources;
wherein said cannabinoid extract is composed of at least about 95% cannabidiol by weight, and
wherein said cannabinoid extract is essentially free from tetrahydrocannabinol (THC) components.

2. The composition of claim 1, wherein the cannabinoid extract further comprises cannabigerol (CBG), cannabidivarin (CBDV), Cannabinol (CBN) and/or cannabichromene (CBC).

3. The composition of claim 1, wherein the cannabinoid extract is composed of at least about 96%, 97%, 98% or 99% cannabidiol by weight.

4. The composition of claim 1, wherein the cannabinoid extract further comprises less than 1% (CBG), (CBDV), (CBN) and/or cannabichromene (CBC) by weight.

5. The composition of claim 1, wherein the phospholipid is phosphocholine (PC), egg PC, soybean PC, DPPC or DOPC.

6. The composition of claim 1, wherein the liposomes of the liposomal formulation are composed of at least 75%, 80% or 85% PC.

7. The composition of claim 1, wherein the liposomes comprise a cholesterol component.

8. The composition of claim 1, wherein the liposomes of the liposomal formulation further comprise a protein component, BSA and/or lithocholic acid.

9. The composition of claim 1, wherein the aqueous carrier comprises water, fruit juice, vegetable juice or an alcohol.

10. The composition of claim 1, wherein the liposomes of the liposomal formulation have an average diameter of between 85 nm and 95 nm.

11. The composition of claim 1, wherein the liposomes of the liposomal formulation have an average diameter of between 80 nm and 100 nm and are composed of at least 80% PC.

12. The composition of claim 1, wherein the composition further comprises curcumin and/or ginger extracts.

13. The composition of claim 1, wherein the composition further comprises melatonin and/or magnesium.

14. The composition of claim 1, wherein the composition further comprises melatonin and/or magnesium.

15. The composition of claim 1, wherein the liposomal formulation comprises omega-3 fatty acids.

16. The composition of claim 1, wherein the composition comprises an oil component comprising canola oil, flaxseed oil, rapeseed oil, soybean oil, walnut oil, fish oil, safflower oil, chia seed oil, sunflower seed oil, sesame seed oil, seaweed oil, corn oil, cotton seed oil, peanut oil, palm oil, avocado oil, coconut oil, or olive oil.

17. The composition of claim 1, formulated as an ingestible tincture, beverage, shot, capsule, tablet or gummy; or as a cream for topical administration.

18. A method treating of treating inflammation in a subject comprising administering the composition of claim 1: wherein the inflammation is skin inflammation, wound inflammation, cardiovascular inflammation, neurological inflammation, liver inflammation or gut inflammation; and wherein the administration of said composition is effective to reduce levels of pro-inflammatory cytokines in the subject; and wherein the administration is effective to increase IL-10 levels, reduce neutrophil accumulation, reduce IL-1β levels and/or reduce TNFα levels within blood or tissues of the subject.

19. A method for treating or preventing a neurological disease or neurological injury in the subject comprising administering the composition of claim 1: wherein the administration of said composition is effective to reduce levels of pro-inflammatory cytokines in the subject; and wherein the administration is effective to increase IL-10 levels, reduce neutrophil accumulation, reduce IL-1β levels and/or reduce TNFα levels within blood or tissues of the subject; and further wherein the administration of said composition reduces beta-amyloid levels in the subject.

20. The method of claim 19 wherein said neurological disease or neurological injury is a stroke or Alzheimer's disease.
